(19) United States
(12) Patent Application Publication
(10) Pub. No.: US 2026/0102081 A1
(43) Pub. Date: Apr. 16, 2026

(54) ANALYTE SENSOR MEMBERS WITH DELAYED SENSOR ACTIVATION

(71) Applicant: Laxmi Therapeutic Devices, Inc., Goleta, CA (US)

(72) Inventors: William Peter Van Antwerp, Santa Clarita, CA (US); Peter Rule, Los Altos Hills, CA (US); Thomas Metzmaker, Goleta, CA (US)

(21) Appl. No.: 19/355,054

(22) Filed: Oct. 10, 2025

Related U.S. Application Data
(60) Provisional application No. 63/705,780, filed on Oct. 10, 2024.

(51) Int. Cl. A61B 5/145; A61B 5/00

(52) U.S. Cl. CPC A61B 5/14532; A61B 5/6802

(57) ABSTRACT

A monitor for determining analyte concentrations in vivo includes a housing configured to adhere to a patient’s skin, and a sensor member configured to extend from the housing into the patient’s skin. The sensor member includes at least a first working electrode and a separate second working electrode that are configured to be activated at different times upon implantation of the sensor member into the patient’s skin.

### CROSS-REFERENCE TO RELATED APPLICATIONS

The present application claims priority to and the benefit of U.S. Provisional Patent Application No. 63/705,780, filed October 10, 2024, the contents of which are hereby incorporated by reference in their entirety.

### BACKGROUND

### Field

The present disclosure relates to medical devices such as analyte sensors, which can be used to measure analytes, such as glucose levels, in the body of a subject. More specifically, the present disclosure is directed to improvements in sensor members associated with such medical devices, as well as methods of use and methods of manufacturing such improved sensor members.

### Description of Related Art

Monitoring different analytes in the human body can be used for various diagnostic reasons. In particular, monitoring glucose levels is important for individuals suffering from type 1 or type 2 diabetes. People with type 1 diabetes are unable to produce insulin or produce very little insulin, while people with type 2 diabetes are resistant to the effects of insulin. Insulin is a hormone produced by the pancreas that helps regulate the flow of blood glucose from the bloodstream into the cells in the body where it can be used as a fuel. Without insulin, blood glucose can build up in the blood and lead to various symptoms and complications, including fatigue, frequent infections, cardiovascular disease, nerve damage, kidney damage, eye damage, and other issues. Individuals with type 1 or type 2 diabetes need to monitor their glucose levels in order to avoid these symptoms and complications.

Analyte monitors, and in particular, glucose monitors for the monitoring of glucose levels for the management of diabetes, are constantly being developed and improved. Although there are several platforms for monitoring analytes such as glucose available on the market, there is still a need to improve their precision, wearability, and accessibility to end-users. In addition, there is a desire to provide robust, less painful, less error-prone, and/or generally more effective continuous glucose monitors which may be attached to the patient’s body for a more prolonged period of time, as well as glucose monitor features that can be used together with such improved monitor and monitor designs.

In particular, as continuous glucose monitors improve, for example, as life cycles of monitors have proposed to increase in certain applications and as various different ways have been proposed to provide increased accuracy and decreased discomfort for patients who wear and use such monitors, it has become more difficult to develop sensor members for such continuous glucose monitors that satisfy all of these heightened expectations.

More specifically, one particular limitation to increasing the functional life span of continuous glucose monitors has been limitations on how long a sensor member of the monitor, or more particularly, the electrodes or active region of the sensor member, is able to provide accurate readings that can be effectively converted into useable data about the patient or end user. Generally, analyte sensor life is limited by, among other factors, the available total enzyme provided on the sensor and the immunological response of the patient. Immune responses are natural occurrences that arise in part due to the presence of the sensor member itself, associated enzymes, and other foreign material simply being in the skin, where they are intrinsically treated as irritants or foreign bodies/materials by the patient’s immune system. Due to these factors, among others, a typical sensor member may only effectively gather data for 14 days or less, which leads existing manufacturers to require typical continuous glucose monitors be changed or switched out at least every two weeks or 14 days, if not sooner.

### SUMMARY

Many continuous glucose monitors are intended to be worn on a patient’s skin for a duration of multiple days or weeks. Most or all commercially available glucose sensors on the market today sense glucose in interstitial fluid (ISF) below the surface of the skin. Such sensing or monitoring therefore typically involves an initial step of inserting a sensing portion of a sensor member of the glucose monitor under the patient’s skin. For the most part, this insertion step will involve puncturing the surface of the skin, for example, with a separate needle, for example, on an applicator to provide access for inserting the sensor. Thereafter, the needle or other sharp may be retracted, while the sensor member stays in place under the patient’s skin. The rest of the monitor may be adhered to the patient’s skin above the sensor member, and may be physically connected to the sensor member, in order to hold the sensor member in place under the skin, to protect the sensor member from environmental and other external conditions, and to provide and house electronics associated with the analyte monitoring, for example, a battery to provide power to the system, a processor and other circuitry to record and/or analyze electrical signals received from the sensor member, and/or a transmitter to transmit data to an offsite location, for example, a cellular phone or server configured to receive and further process the collected data.

Continuous glucose monitors and sensors need to be very consistently accurate, in order to perform their function of detecting blood glucose levels for a patient seeking to self-medicate with insulin. Such continuous glucose monitors are relied upon to alert the patient of low blood glucose levels, in which case the patient can administer insulin to raise blood glucose levels and alleviate the issue. Therefore, an inaccurate reading could potentially lead to patient harm, for example, failing to alert the patient to administer insulin when the patient’s blood glucose levels are low, or improperly alerting the patient of low blood glucose and causing the patient to administer insulin when it is not medically necessary to do so.

In an effort to improve monitoring, reliability, and robustness, continuous glucose sensors have been met with a number of different challenges. Among these challenges have been, for example, restrictions on sensor life and efficacy due to the amount of enzyme available to facilitate the necessary enzymatic reactions associated with monitoring, time delays associated with the measurements taken by the monitors as compared to the actual blood glucose levels of the patient as well as the variability of such delays with respect to the depth and/or other positioning of the sensing regions of the sensor members, natural or other biological responses causing biofouling (e.g., the accumulation of unwanted biological material on the sensor member or surroundings that may cause unwanted deviations in the monitor’s efficacy), and/or changes in position of the sensor member due to motion, for example, due to movement by the patient.

In addition, continuous glucose sensors must maintain robust electrical connections between their sensing regions and the rest of the electronics in the housing, with the electrical connections being strong enough to sustain through the life of the sensor, so that data can be properly acquired from the sensing regions. However, these robust electrical connections cannot adversely affect the performance of the sensing regions themselves. So a more effective way to provide both consistent and robust electrical connections has also been sought.

And lastly, as noted above, the effectiveness of sensor members deteriorate over time due to many different factors, to the extent that needing to replace sensor members due to deteriorating performance or the threat of such has become a major factor or limitation that has prevented manufacturers from effectively increasing the life span of continuous glucose monitors in general.

According to embodiments of the invention, sensor members may be fabricated or otherwise manufactured with two or more working electrodes, or two or more sets of electrodes (e.g., with multiple working electrodes and associated reference electrodes and/or counter electrodes). At least the working electrodes in such multiple electrode arrangements would generally function independently from one another. Such multiple electrodes can be arranged in different ways on the sensor member, depending on the desired performance characteristics and features.

In such arrangements, a first working electrode or first set of electrodes may be active initially upon implantation, while exposure, activation, or other triggering mechanism to initiate functionality of the remaining working electrode(s) or set(s) of electrodes may be intentionally delayed using one or more different arrangements or methods. Such additional working electrodes or sets of electrodes may initially further be covered, insulated, or otherwise prevented from exposure to the skin, so that general deterioration of the additional electrodes can be minimized or prevented. Thereafter, when the first working electrode or first set of electrodes is nearing end-of-life, a second working electrode or set of electrodes can be activated to replace the functionality of the first working electrode or first set of electrodes. Timing can be based on, for example, a preset duration, or may involve active monitoring of the performance characteristics of the active working electrode or set of electrodes, or a combination of these two and/or other factors. Under such an arrangement, the analyte monitor can continue working even if the active regions of the first sensor member stop working or are otherwise turned off, and the effective life of the analyte monitor as a whole can be increased.

According to an embodiment of the invention, a monitor for determining analyte concentrations in vivo includes a housing configured to adhere to a patient’s skin, and a sensor member configured to extend from the housing into the patient’s skin. The sensor member includes at least a first working electrode and a separate second working electrode that are configured to be activated at different times upon implantation of the sensor member into the patient’s skin.

The first and second working electrodes may be independently connected to electronics in the housing via separate electrical connections.

The first and second working electrodes may be sufficiently spaced apart from one other such that the second working electrode is configured to avoid an immune response to operation of the first working electrode.

The monitor may further include a coating layer configured to cover the second working electrode while remaining spaced apart from the first working electrode. The coating layer may be inert. The coating layer may include a material that is configured to decay when exposed to glucose, and the material may include a glucose responsive polymer. The coating layer may include a material that is removable upon applying a sufficient current thereacross, the coating layer may include gold, and/or a trace may connect the coating layer to the electronics in the housing. The coating layer may be removable from the sensor member via application of a sufficient shear force thereto.

The first and second electrodes may be respectively arranged on opposite faces of the sensor member.

The first and second working electrodes may both be configured to be positioned away from the subcutaneous layer of the patient’s skin.

The monitor may further include a first counter electrode and a first reference electrode utilized by the first working electrode, and a second counter electrode and a second reference electrode utilized by the second working electrode.

When the first working electrode fails, otherwise stops working, or is determined by the monitor to be near failure, the monitor may activate the second working electrode to replace the first working electrode.

The second working electrode may be configured to be activated at least one week after the first working electrode is activated.

According to another embodiment of the invention, a monitor for determining analyte concentrations in vivo includes a housing configured to adhere to a patient’s skin, and a sensor member configured to extend from the housing into the patient’s skin. The sensor member includes at least a first working electrode, a separate second working electrode, and a coating layer configured to cover the second working electrode while remaining spaced apart from the first working electrode.

The coating layer may include a glucose responsive polymer that is configured to decay when exposed to glucose.

The coating layer may include a material that is removable upon applying a sufficient current thereacross.

The coating layer may be inert.

According to embodiments of the invention, continuous glucose monitors may be allowed to have significantly longer life, and will not be limited due to the limited life of the active regions of sensor members. If a typical active region of a sensor member lasts approximately two weeks, the life expectancy of a sensor member according to embodiments of the invention will at least be doubled in cases where there is a second working electrode or second set of electrodes, and may be increased even more for sensor members that incorporate even more working electrodes or sets of electrodes with delayed exposure or activation.

### BRIEF DESCRIPTION OF THE DRAWINGS

Further features and advantages of the invention will become apparent from the description of embodiments by means of the accompanying drawings. In the drawings:

FIGS. 1A and 1B schematically show a human body with an analyte monitor including an analyte sensor according to embodiments of the invention, where the analyte monitor is attached at different positions on the body.

FIG. 2 shows a perspective view from above an exemplary analyte monitor including an analyte sensor according to embodiments of the invention.

FIG. 3 shows a perspective view from below the analyte monitor of FIG. 2.

FIG. 4 shows a cutaway view of a portion of a patient’s skin with a schematic depiction of a sensor member of an analyte monitor according to an embodiment of the invention implanted therein.

FIG. 5 shows an enlarged view of a portion of a sensor member of an analyte monitor according to a first embodiment of the invention.

FIG. 6 shows an enlarged view of a portion of a sensor member of an analyte monitor according to a second embodiment of the invention.

FIG. 7 shows an enlarged view of a portion of a sensor member of an analyte monitor according to a third embodiment of the invention.

FIG. 8 shows an enlarged view of a portion of a sensor member of an analyte monitor according to a fourth embodiment of the invention.

### DETAILED DESCRIPTION

In the following detailed description, only certain embodiments of the subject matter of the present disclosure are described, by way of illustration. As those skilled in the art would recognize, the subject matter of the present disclosure may be embodied in many different forms and should not be construed as being limited to the embodiments set forth herein.

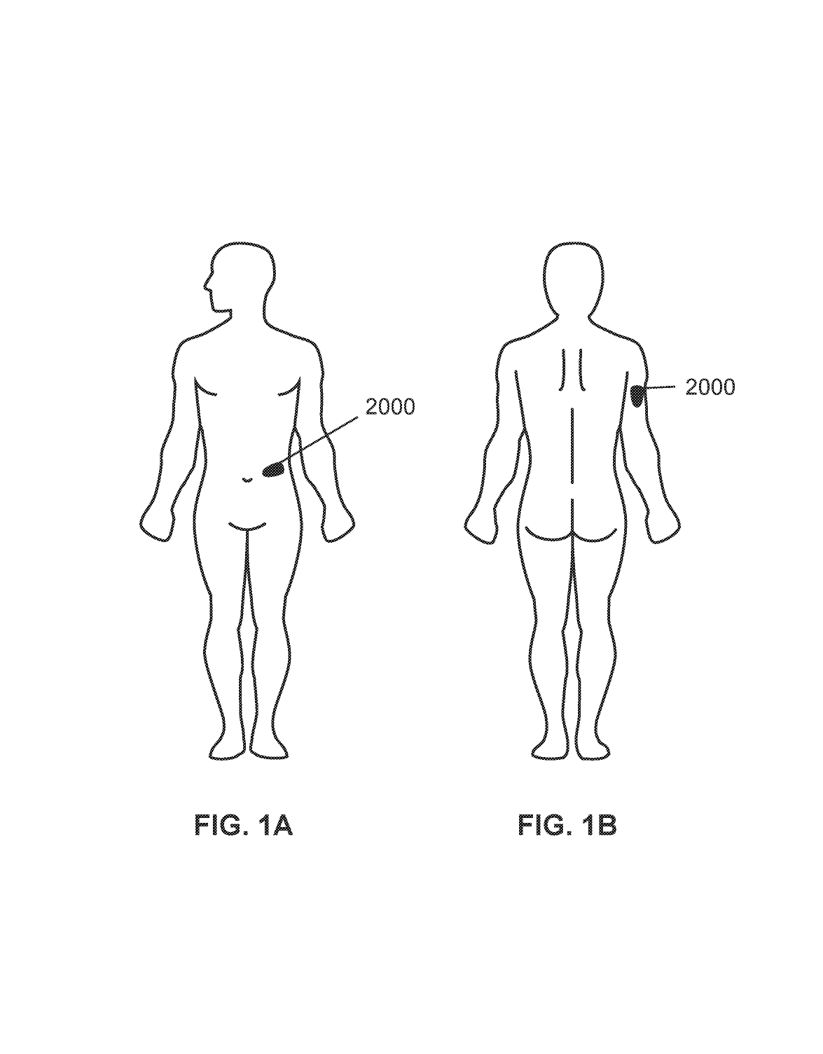

Monitors that include analyte sensors, such as glucose monitors, and in particular continuous glucose monitors, can be attached to a patient’s body in different locations, in order to for example, improve glucose monitoring and/or a patient’s comfort, since the continuous glucose monitors must remain adhered to the patient’s skin, sometimes for a few days or more. FIG. 1A shows a first exemplary analyte monitor 2000 that is adhered to a patient’s abdominal region, while FIG. 1B instead shows the exemplary analyte monitor 2000 adhered to a patient’s arm. These are only meant to be example adhesion sites, and in other situations, this or a similar analyte monitor may instead be adhered or otherwise attached to other parts of the patient’s body.

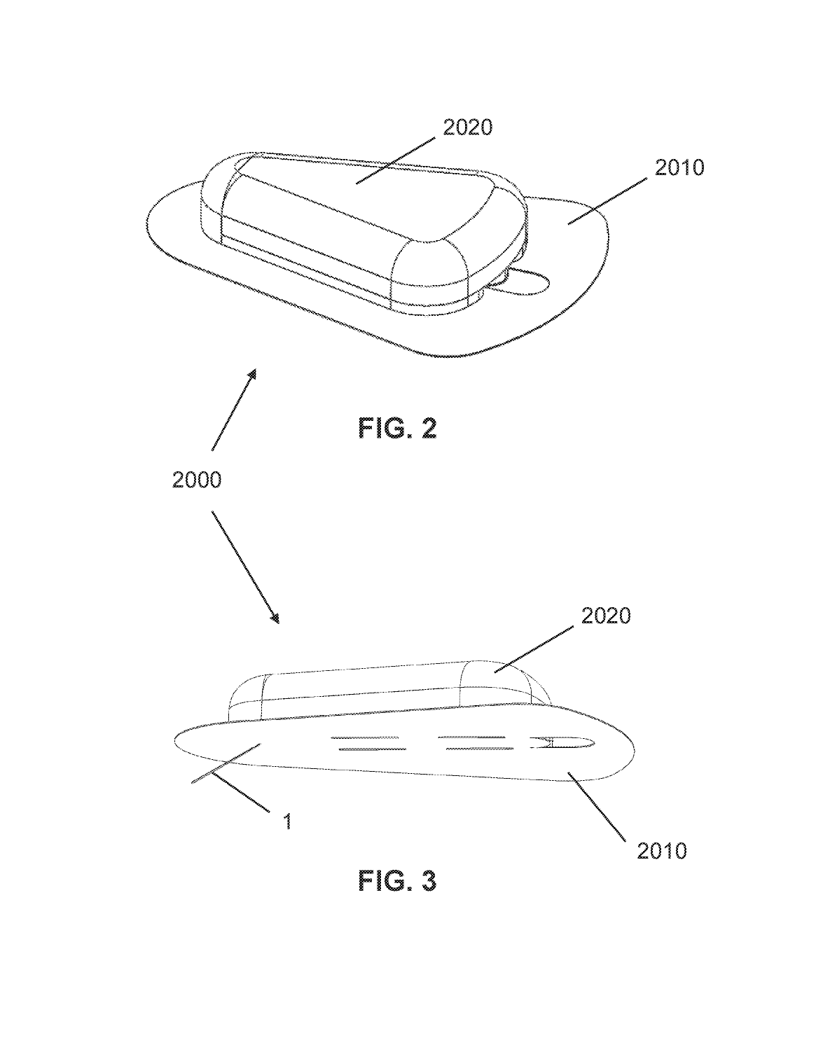

FIGS. 2 and 3 show different schematic views of an example analyte monitor 2000, which can be a continuous glucose monitor, according to an embodiment of the invention. The continuous glucose monitor 2000 may include a base or cradle 2010 that may have an adhesive layer for adhering to a patient’s skin, a transmitter 2020 for transmitting data to and/or from a location away from the monitor, and a sensor member 1 which may include an integrated analyte sensing region such as a glucose sensor. It is to be understood that the example analyte monitor shown in FIGS. 2 and 3 are for illustrative and descriptive purposes only, and that analyte monitors with different structures and functionality can also be used in conjunction with the sensor assemblies described below, without departing from the spirit or scope of the invention.

The base 2010 will generally include an adhesive patch or other adhesive mechanism on its lower surface in order to facilitate attachment of the base 2010 to the surface of the patient’s skin. In some embodiments of the invention, the sensor member 1 may be integrally formed with the base 2010 so as to be implanted under the patient’s skin upon attachment of the base 2010 to the surface of the skin, while in other embodiments the base 2010 may be applied to the skin first, and the sensor member 1 may be advanced to a position where the sensor member 1 becomes attached to the base during or after implantation of part of the sensor member 1 under a patient’s skin. For some embodiments, application of the base 2010 and/or the sensor member 1 may be facilitated with one or more applicators.

In some embodiments, after the base 2010 has been attached to a surface of the patient’s skin and after at least part of the sensor member 1has been advanced under the patient’s skin, the transmitter 2020 which may be a separate part can be attached to the base. In some embodiments, the transmitter 2020 may include, for example, a power source such as a battery, while the base 2010 and/or the sensor member 1may include additional electrical circuitry or contacts to complete a circuit, such that the monitor 2000 is powered up upon assembly of the transmitter 2020 to the base 2010. Other embodiments may include other arrangements, for example, where the battery is housed in the base 2010 instead of on the transmitter 2020, etc. Still other embodiments may include further different arrangements, for example, an integrally manufactured monitor where the base and transmitter are formed together in a single main body and which may not be separable from one another by the end user, where such embodiments may also include an integrated sensor member, or may include a sensor member that is implanted through the main body of the monitor after the main body has been adhered to the surface of the patient’s skin.

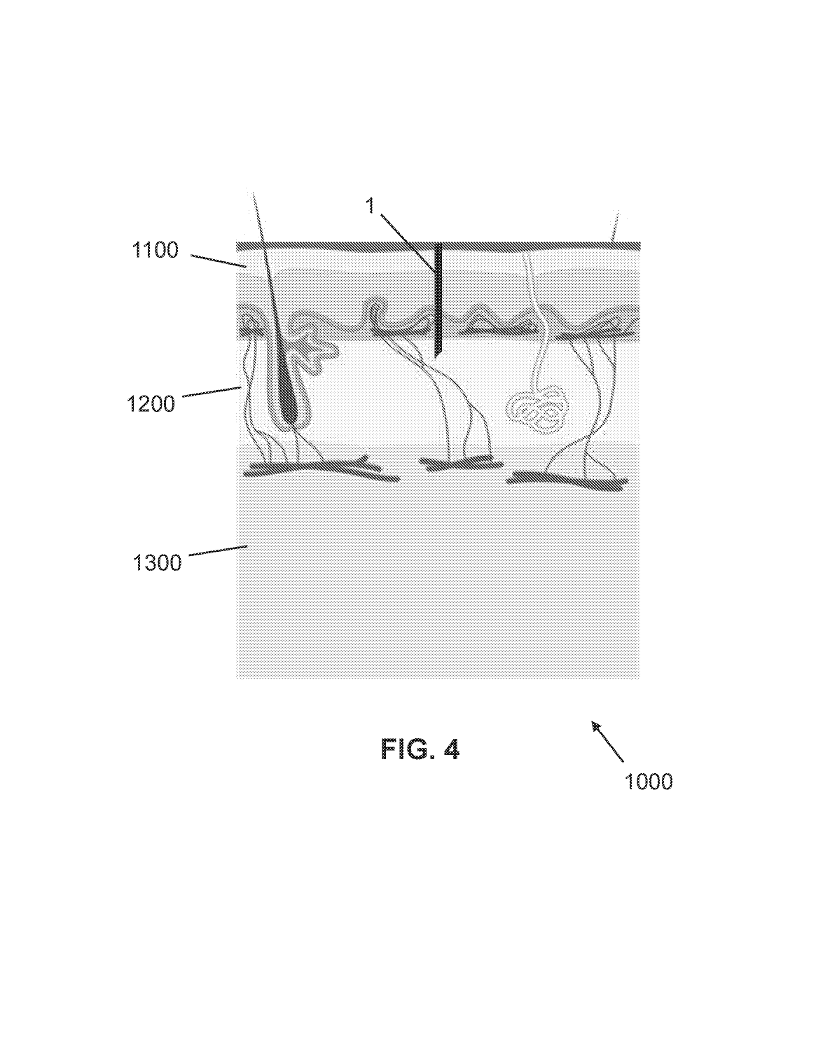

FIG. 4 shows a cutaway view of a portion of a patient’s skin, with a schematic depiction of the sensor member 1 implanted therein, according to an embodiment of the invention. As shown in FIG. 4, the skin 1000 of a human includes various layers, such as an epidermis 1100 that is closest to the surface of the skin, an intermediate dermis 1200, and a deeper hypodermis or subcutaneous layer 1300. Sensor members 1 according to embodiments of the invention may only extend into the shallow regions of the dermis 1200, with sensing regions in either the shallow regions of the dermis 1200, the epidermis 1100, or both, rather than extending more deeply into the hypodermis 1300 like many other monitors on the market. Positioning the sensing regions of the sensor member 1 in the shallow regions of the dermis 1200 or the epidermis 1100 may have many advantages. For example, the smaller size of the sensor member 1 according to embodiments of the invention, both in terms of length and thickness, may reduce trauma during insertion and/or discomfort during the life of the sensor/monitor. The reduction in size of the sensor member 1 compared to traditional sensor members, or more generally, the reduced depth to which the sensor member 1 extends under the skin, will also allow sensing in less fatty layers of the skin, consequently resulting in faster glucose transport, more accurate signal readings by the sensor member, and a shorter delay or time lag associated with being able to retrieve useable signals from the sensor member 1. Targeting the shallower regions of the dermis 1200 furthermore allows for some tolerance or leeway, where for example, if a sensor member 1 is implanted at a slightly more vertical angle than intended, the sensing regions of the sensor member 1 should still be positioned in the dermis 1200 rather than deeper in the hypodermis 1300, and still benefit from both reduced discomfort and less glucose transport delays due to fat in the tissue.

Sensor members in other embodiments may be positioned shallower or deeper than as described, without departing from the spirit or scope of the invention. For example, as has been previously discussed, some embodiments of the invention may incorporate multiple working electrodes or electrode sets or bundles that are spaced apart along a length or axis of extension of the sensor member. In such embodiments, the sensor member may be arranged to be longer, for example, configured to extend into the hypodermis 1300. However, at least one working electrode or electrode set may be arranged more proximally or shallower on the sensor member, so as to be positioned in the epidermis 1100 or shallow dermis 1200, for example, when the sensor member is fully implanted, while a further electrode or electrode set may be arranged more distally or deeper on the sensor member, so as to be positioned in the hypodermis 1300, for example, when the sensor member is fully implanted. Such an arrangement may allow for collection of different types of signals, which may be beneficial in some signal processing cases or situations, as described in greater detail below.

In yet other embodiments, the body of the sensor member may be constructed to be longer, for example, to extend into the hypodermis 1300, while the electrodes or other sensing regions may all still be configured to be positioned shallower in the dermis 1200 or epidermis 1100. Here, the extra extension of the body of the sensor member may help to facilitate a more robust anchoring of the sensor member under the patient’s skin. In still other embodiments, the body of the sensor member may be constructed longer, but may be configured to be implanted under the patient’s skin at an angle, so that despite its longer length, the entire sensor member, or at least the distal portions thereof where a more distally located electrode(s) or sensing region(s) is located, will still be positioned in the dermis 1200 upon implantation. Any of various other arrangements may also be employed, without departing from the spirit or scope of the invention.

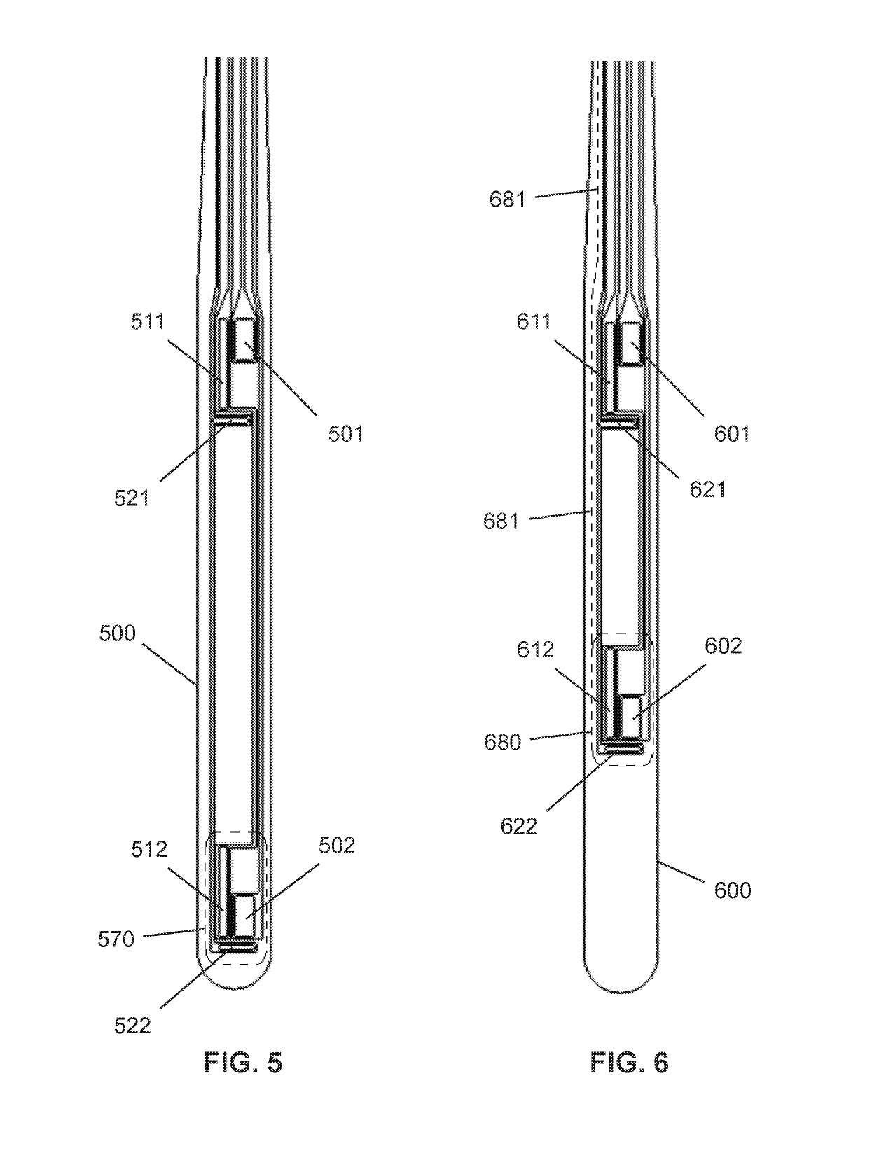

Construction of an example sensor member according to a first embodiment of the invention will now be discussed in greater detail, with reference to FIG. 5. The portion of the sensor member shown in FIG. 5 may be a distal end portion of the sensor member. As seen in FIG. 5, the sensor member may include an implantable portion that includes a main body 500 that may extend from a more proximal end (the upper end as illustrated) that interfaces with a monitor housing (not shown) to a distal end (the lower end as illustrated). The main body 500 of the sensor member may be elongate, as previously discussed, and in some embodiments, may further include one or more retention mechanisms or means, for example, barbs or cutouts along sides of the body, different shapes such as an enlarged distal tip region, and/or roughened surfaces. In addition, as previously discussed, while the main body 500 in FIG. 5 is illustrated as being generally elongate in shape, the electrodes or other sensing regions may still nevertheless be configured to be positioned in the dermis or epidermis of the skin, instead of the subcutaneous region. Longer sensors members such as the one shown in FIG. 5 can realize this, for example, by be implanted at an angle relative to the surface of the skin. In this manner, a longer sensor member can be implanted under the skin, reducing the likelihood of inadvertent withdrawal of the sensor member, while the sensing regions still remain in more shallow regions of the patient’s skin. Meanwhile, in some embodiments, the second sensing region may be positioned on the sensor member to be intentionally positioned in the subcutaneous layer, and sensing characteristics for the second sensing region can be adjusted or otherwise compensated accordingly.

The main body 500 may include two sensing regions. A first sensing region may include a first working electrode 501, a first counter electrode 511, and a first reference electrode 521. Meanwhile, a second sensing region may include a second working electrode 502, a second counter electrode 512, and a second reference electrode 522. The sensor member shown in FIG. 5 is arranged such that the first sensing region is more proximally positioned than the second sensing region, with the second sensing region positioned at or near a distal tip of the main body 500. The respective working electrodes 501, 502 may work together with their respective counter electrodes 511, 512, and their respective reference electrodes 521, 522, to generate signals that can be processed to determine blood glucose levels of the patient in which the sensor member is implanted. The electrode sets, according to embodiments of the invention, may be fabricated or otherwise manufactured, for example, by leveraging improved photolithography and/or other compatible nano-manufacturing technology methods, and may be arranged, for example, with a layered filament design.

In addition, there is a large gap or space between the two sensing regions, and in particular, between the first working electrode 501 and the second working electrode 502, so as to ensure electrical isolation of the electrode sets from one another. Such an arrangement can ensure that data is collected by the respective sets of electrodes completely separately from one another. Another reason to provide sufficient spacing between the sets of electrodes or sensing regions is to avoid one set of electrodes being subject to the immune response associated with the other set of electrodes. Both the first set of electrodes and the second set of electrodes have electrical connections that extend upward (as illustrated) and out above the surface of the skin to electrically connect with electronics that are housed in a housing of a monitor (not shown) that may be adhered to the surface of the patient’s skin.

As shown in FIG. 5, one or more electrodes from among the second set of electrodes 502, 512, 522 may further be covered by an inert coating 570, which may be removed at a later time. In the embodiment of FIG. 5, the entire second sensing region, including the working electrode 502, the counter electrode 512, and the reference electrode 522, are fully covered by the inert coating 570. Such an arrangement allows the second set of electrodes to be activated at a later time, for example, when the first set of electrodes 501, 511, 521 run out of enzyme, are no longer effective due to immune response in the area surrounding the first set of electrodes, general wear and tear, and/or due to any other factors or reasons.

In FIG. 5, the inert coating 570 is an isolated coating, which may be realized by a general deposition process where a material is simply coated over the entire second sensing region, just the second working electrode 502, or any subset of the second sensing region, inclusive or exclusive of other electrodes, so long as the second working electrode 502 is covered by the inert coating 570. In such a manner, upon implantation, the system will include two or more sets of electrodes, with a first set of electrodes 501, 511, 521 initially exposed to the patient’s interstitial fluid, while the second set of electrodes 502, 512, 522 is kept or otherwise preserved behind a barrier that prevents diffusion to and from the electrodes. Some arrangements may further include additional electrodes or electrode sets, for example, a third electrode set (not shown) that may be fabricated on an opposite side of the main body 500 of the sensor member. In such an arrangement, the other set(s) of electrodes may become exposed to the patient’s interstitial fluid at a later time, for example, after a triggering mechanism or a predetermined time delay. In cases where the inert coating 570 is provided without any further electrical connections, as shown in FIG. 5, the inert coating 570 might be realized, for example, by a glucose responsive polymer. The glucose responsive polymer may initially cover, protect, and electrically isolate the second set of electrodes 502, 512, 522, and may deteriorate over time after a certain total glucose exposure. The desired time delay in such an arrangement may be controlled, for example, by controlling the thickness of the glucose responsive polymer that covers the second set of electrodes, such that the glucose responsive polymer will slowly decay over time when exposed to the naturally occurring glucose in the patient’s interstitial fluid. Here, the second set of electrodes may be exposed to the interstitial fluid only after the glucose responsive polymer is exposed to a certain amount of glucose, which can be easily controlled by the manufacturer. One example of a mechanism that can control the thickness of a fabricated layer of glucose responsive polymer might be using slot coating or a spray coater in combination with a shadow mask. Other examples may also be used, so long as a desired thickness of glucose responsive polymer can be consistently applied to sensor members during manufacturing. In addition to glucose responsive polymers, other coverings or coatings can also be used, with one particular alternative example discussed in greater detail below, with respect to the embodiment shown in FIG. 6.

FIG. 6 shows an example of a portion of a sensor member according to a second embodiment of the invention. The portion of the sensor member shown in FIG. 6 may be a distal end portion of the sensor member. As seen in FIG. 6, the sensor member may include an elongate main body 600, with sets of electrodes formed on the sensor member that are arranged similarly to the sets of electrodes on the sensor member in FIG. 5. The sensing regions may include a more proximally positioned first sensing region or first set of electrodes, including a first working electrode 601, a first counter electrode 611, and a first reference electrode 621, and a more distally positioned second sensing region or second set of electrodes, including a second working electrode 602, a second counter electrode 612, and a second reference electrode 622. The sets of electrodes formed in the sensing regions of the sensor member in FIG. 6 will generally function similarly to the sensing regions of the sensor member in FIG. 5. However, while the first sensing region in the sensor member of FIG. 6 is arranged on the main body 600 in a substantially similar position as the first sensing region is arranged on the main body 500 in FIG. 5, the second sensing region including second working electrode 602, second counter electrode 612, and second reference electrode 622 is positioned farther away from the distal tip of the main body 600, and therefore closer to the first set of electrodes 601, 611, 612. While there is still a gap between the first and second sensing regions, the gap is smaller than the gap on the sensor member in FIG. 5. Such an arrangement may more easily accommodate implantation of the sensor member at an angle perpendicular to a surface of the patient’s skin, or at an angle closer to perpendicular, where a portion (or a larger portion) of the main body can extend into the subcutaneous layer of the patient’s skin, while the second sensing region is configured to remain in the dermal layer of the skin.

In addition, the second more distally located sensing region of the sensor member may be covered by an inert coating 680, similarly as seen with the sensor member in FIG. 5. In FIG. 6, the inert coating 680 may further be electrically connected to other electrical components in the monitor housing via an additional trace 681. In the embodiment shown, the inert coating 680 may be made of a material that can be removed by applying a current across the material. One such material may be gold, but other biocompatible materials may be contemplated and utilized as well. In such arrangements, a sufficiently strong current may be passed through trace 681 at a desired time, in order to pass a current through the inert coating 680 and thereby remove the inert coating 680. The trace 681 may create an electrical connection to remove the coating at a specified time (typically more than one week after initial activation, for example, 10 days or two weeks after), or may be triggered in reaction to an event, such as the first electrode set or another electrode set no longer functioning or functioning correctly. The inert coating 680 may be applied using, for example, a sputtering process with a shadow mask that controls where the coating is deposited, or by any other appropriate method.

While two specific types of inert coating have been described in the prior examples, other methods of removing the inert coating may be employed in still other embodiments. Aside from general decay of the inert material over time or applying a current across the inert material to remove the material, another example might be to remove the inert coating using a mechanical mechanism, such as by applying a shear force on the inert coating or on another layer of material adjacent the inert coating, or both, where moving two layers relative to one another with a sufficient force or speed may cause the layers to shear or otherwise separate from one another. Any of various other methods may also be employed without departing from the spirit or scope of the invention.

In addition, while the electrode arrangements shown in FIGS. 5 and 6 are provided similarly, it is to be understood that other electrode and electrode set arrangements may also be employed, without departing from the spirit or scope of the invention. For example, in some embodiments, the first and second sensing regions may be positioned much closer to one another, for example, directly adjacent to one another. In addition, while the inert coating covers the more distally positioned sensing region in both described embodiments, other embodiments may instead initially cover the more proximally positioned sensing regions instead, such that the more distal sensing region is activated first, while the more proximal sensing region is activated at a later time instead.

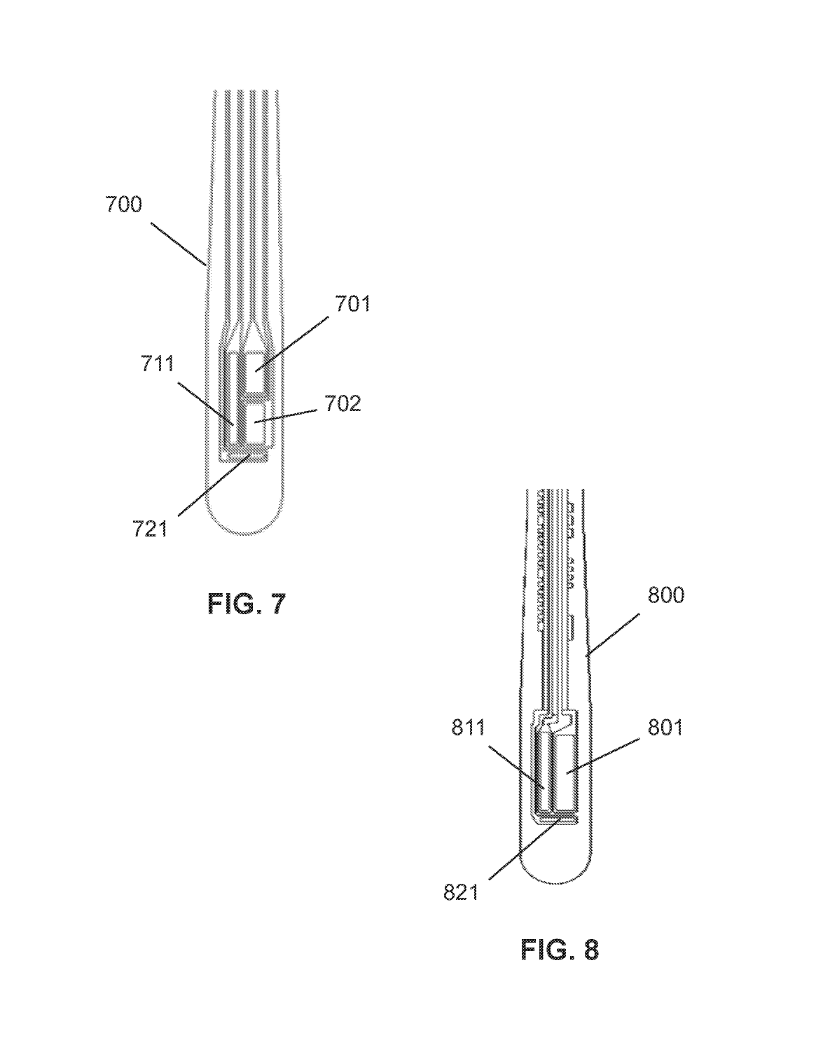

FIG. 7 shows an example of a portion of a sensor member according to a third embodiment of the invention. The portion of the sensor member shown in FIG. 7 may be a distal end portion of the sensor member. The sensor member in FIG. 7 is presented to provide an alternative electrode arrangement. It is to be understood that delay of one of the first or second working electrodes 701, 702 may be realized similarly to the previous embodiments, and so discussion of the delay mechanisms in this embodiment, as well as in the embodiment shown in FIG. 8, have been omitted and will not be repeated.

As seen in FIG. 7, the set of electrodes on the main body 700 of the sensor member includes a first working electrode 701, a single counter electrode 711, and a single reference electrode 721. However, the sensing region on the sensor member in FIG. 7 further includes a second working electrode 702 integrated into the same set of electrodes as the first working electrode 701, such that the first and second working electrodes 701, 702, share the same counter electrode 711 and the same reference electrode 721. The working electrodes 701, 702 may be functionally equivalent but function independently from one another, and may include separate respective leads to facilitate separate communication between the working electrodes 701, 702, and the rest of the electronics package of the analyte sensor, for example, housed in the external portion of the monitoring system. Separate construction of the working electrodes 701, 702 also ensures that the working electrodes 701, 702 can properly function independently from one another. In other arrangements, the working electrodes 701, 702 may be arranged in series or in parallel, for example, depending on the particular needs and/or objectives of the sensor member.

As illustrated, the sensor member includes one counter electrode 711 and one reference electrode 721 that work together with the two working electrodes 701, 702, to monitor analyte levels such as blood glucose levels in the patient. In other embodiments, there may also be more than one counter electrode and/or reference electrode. For example, in some embodiments, there may be a separate corresponding counter electrode for each working electrode, which for the example shown in FIG. 7, may be arranged such that each working electrode and corresponding counter electrode are positioned adjacent to one another in a width direction. Such additional electrode arrangements may also be wired independently, in series, or in parallel, or may also be otherwise arranged. Still other arrangements or electrodes and/or electrode sets may also be incorporated without departing from the spirit or scope of the invention.

FIG. 8 shows an example of a portion of a sensor member according to a fourth embodiment of the invention. The portion of the sensor member shown in FIG. 8 may be a distal end portion of the sensor member. The sensor member in FIG. 8 also includes a main body 800, with a first face on which a first sensing region is formed, with the first sensing region including a first working electrode 801, a first counter electrode 811, and a first reference electrode 821. While not visible in FIG. 8, a second sensing region with a second set of electrodes, such as a second working electrode, a second counter electrode, and a second reference electrode, may be formed on a back or opposite face of the main body 800, e.g., on a side of the main body 800 that faces the paper in FIG. 8. Here, activation of the sensing region on one of the faces of the sensor member may be delayed, similarly as discussed in previous embodiments. Such an arrangement can be manufactured in any of various different methods, for example, by adhering or otherwise attaching two fabricated sensors together back-to-back, by folding a fabricated sensor in half, or by otherwise modifying the fabrication process to facilitate backside fabrication.

While not pictured in detail, sensor members according to some or all embodiments of the invention can be formed on a thin flexible substrate, to facilitate less trauma to the surrounding tissue and less discomfort to the patient both during insertion of the sensor member and while the sensor member is held under the patient’s skin. The sensor member may further be fabricated or otherwise manufactured in layers, for example, with a flexible base or substrate, electrodes and electrical/conductive connections and other materials to facilitate electrical communication with the wearable electronics package housed in the main body of the analyte sensor, enzymatic material to facilitate the electrochemical reactions necessary for a desired type of analyte monitoring, the inert coating or covering layer or layers, a diffusion limiting membrane, and/or a hydrophilic outside layer. More or less other layers may also be incorporated, based on the particular design of the sensor member.

Some other sensors on the market have tried to employ a working electrode connected to multiple locations in series on their respective sensor members, or may include multiple working electrodes that are otherwise operated concurrently, in an effort to, for example, obtain an average signal from multiple tissue spaces. However, such arrangements are not amenable to delaying activation of one or more working electrodes compared to a first activated working electrode. In contrast, the electrode arrangements according to embodiments of the invention provide a new way to delay activation of one or more working electrodes, in an effort to prolong the life span of the monitor as a whole.

Traditionally, measures that have been taken to try to extend the life spans of sensor members of analyte sensors included, for example, polyvinyl alcohol (PVA) coatings and slower diffusion membranes. However, such arrangements cause delays in measurements relative to the actual blood glucose levels of the patient, which may for example, delay treatments in the case of a low blood glucose indication by the system. Other systems have also tried to use a “look forward” algorithm, but such algorithms are based on population data and applied universally, instead of a calibrated comparison more accurately based on data of the specific patient, and therefore is also less accurate. According to embodiments of the invention, a first working electrode can function with less restrictions (e.g., without or with a less prohibitive diffusion layer), and can therefore provide more accurate blood glucose readings with less time delays, while a second working electrode can assume the primary monitoring duties when the first working electrode is nearing end of life.

In addition to the embodiments that have already been described above, it is also possible to combine embodiments, e.g., different features from the various described embodiments, to provide even more different variations of analyte monitors, without departing from the spirit or scope of the invention. In addition, the inventions should not be limited to the structures and/or shapes described in the embodiments above.

While the subject matter of the present disclosure has been described in connection with certain embodiments, it is to be understood that the subject matter of the present disclosure is not limited to the disclosed embodiments, but, on the contrary, the present disclosure is intended to cover various modifications and equivalent arrangements included within the spirit and scope of the appended claims, and equivalents thereof.

What is claimed is:

1. A monitor for determining analyte concentrations in vivo, comprising:
a housing configured to adhere to a patient’s skin; and
a sensor member configured to extend from the housing into the patient’s skin, wherein the sensor member comprises at least a first working electrode and a separate second working electrode that are configured to be activated at different times upon implantation of the sensor member into the patient’s skin.

2. The monitor of claim 1, wherein the first and second working electrodes are independently connected to electronics in the housing via separate electrical connections.

3. The monitor of claim 1, wherein the first and second working electrodes are sufficiently spaced apart from one other such that the second working electrode is configured to avoid an immune response to operation of the first working electrode.

4. The monitor of claim 1, further comprising a coating layer configured to cover the second working electrode while remaining spaced apart from the first working electrode.

5. The monitor of claim 4, wherein the coating layer is inert.

6. The monitor of claim 4, wherein the coating layer comprises a material that is configured to decay when exposed to glucose.

7. The monitor of claim 6, wherein the coating layer comprises a glucose responsive polymer.

8. The monitor of claim 4, wherein the coating layer comprises a material that is removable upon applying a sufficient current thereacross.

9. The monitor of claim 8, wherein the coating layer comprises gold.

10. The monitor of claim 8, wherein a trace connects the coating layer to the electronics in the housing.

11. The monitor of claim 4, wherein the coating layer is removable from the sensor member via application of a sufficient shear force thereto.

12. The monitor of claim 1, wherein the first and second electrodes are respectively arranged on opposite faces of the sensor member.

13. The monitor of claim 1, wherein the first and second working electrodes are both configured to be positioned away from the subcutaneous layer of the patient’s skin.

14. The monitor of claim 1, further comprising a first counter electrode and a first reference electrode utilized by the first working electrode, and a second counter electrode and a second reference electrode utilized by the second working electrode.

15. The monitor of claim 1, wherein when the first working electrode fails, otherwise stops working, or is determined by the monitor to be near failure, the monitor activates the second working electrode to replace the first working electrode.

16. The monitor of claim 1, wherein the second working electrode is configured to be activated at least one week after the first working electrode is activated.

17. A monitor for determining analyte concentrations in vivo, comprising:
a housing configured to adhere to a patient’s skin; and
a sensor member configured to extend from the housing into the patient’s skin, wherein the sensor member comprises at least a first working electrode, a separate second working electrode, and a coating layer configured to cover the second working electrode while remaining spaced apart from the first working electrode.

18. The monitor of claim 17, wherein the coating layer comprises a glucose responsive polymer that is configured to decay when exposed to glucose.

19. The monitor of claim 17, wherein the coating layer comprises a material that is removable upon applying a sufficient current thereacross.

20. The monitor of claim 17, wherein the coating layer is inert.
